(19) United States
(12) Patent Application Publication
(10) Pub. No.: US 2013/0012859 A1
(43) Pub. Date: Jan. 10, 2013

(54) PACKAGED MEDICAL ADHESIVE COMPOSITE AND METHOD OF DELIVERING THE COMPOSITE

(76) Inventor: Amy J. Krohn, Oakdale, MN (US)

(21) Appl. No.: 13/516,080

(22) PCT Filed: Dec. 8, 2010
(86) PCT No.: PCT/US10/59452

§ 371 Date: Jun. 14, 2012

Related U.S. Application Data
(60) Provisional application No. 61/286,538, filed on Dec. 15, 2009.

(51) Int. Cl. A61F 13/02; A61F 13/84; A61B 19/02

(52) U.S. Cl. 602/57; 206/440; 604/385.01

(57) ABSTRACT

Disclosed herein are a packaged medical adhesive composite and a method of delivering the composite. The medical adhesive composite comprises a single tab as a release liner that partially covers the adhesive side of the composite. The tab can be used to apply the composite with only a single hand, even without compromising the sterility of the absorbent pad used to cover a wound. The medical adhesive composite can comprise an especially thin supple backing and be delivered over a wound with little or no wrinkling of the backing. The medical adhesive composite can be delivered such that the composite overlaps with itself when applied, for example, to a finger.

### FIELD

Disclosed herein are a packaged medical adhesive composite and a method of delivering or applying the medical adhesive composite to a patient. The medical adhesive composite may comprise a bandage or wound dressing.

### BACKGROUND

Medical adhesive composites such as bandages, wound dressings, drapes and the like may be packaged to prevent contamination. Among the types of medical adhesive composites are those having a pressure sensitive adhesive layer and absorbent pad disposed on a conformable backing that is thin, flexible and supple. The pressure sensitive adhesive layer is in contact with a release liner. The medical adhesive composite and release liner are packaged between top and bottom packaging sheets that are sealed at the periphery of the packaging sheets around the composite and liner.

### SUMMARY

Disclosed herein are a packaged medical adhesive composite and a method of delivering the composite. The medical adhesive composite comprises a single tab as a release liner that partially covers the adhesive side of the composite. The tab can be used to apply the composite with only a single hand, even without compromising the sterility of the absorbent pad used to cover a wound. The medical adhesive composite can comprise an especially thin supple backing and be delivered over a wound with little or no wrinkling of the backing. The medical adhesive composite can be delivered such that the composite overlaps with itself when applied, for example, to a finger.

The packaged medical adhesive composite may comprise: a medical adhesive composite, a release liner and packaging. The medical adhesive composite may comprise a backing, and a pressure sensitive adhesive layer disposed on the backing; the release liner may be disposed on a portion of the pressure sensitive adhesive layer opposite the backing, such that a portion of the pressure sensitive adhesive layer is not in contact with the release liner; and packaging may comprise: a bottom packaging sheet comprising a bottom substrate and a release layer disposed on the bottom substrate, wherein the portion of the pressure sensitive adhesive layer not in contact with the release liner is in contact with the release layer; and a top packaging sheet disposed on the backing opposite the pressure sensitive adhesive layer, wherein the top and bottom packaging sheets are sealed together about the periphery of the medical adhesive composite and the release liner.

The medical adhesive composite may be delivered over a wound by the following method: providing a packaged medical adhesive composite as described above; at least partially separating the top and bottom packaging sheets; separating the medical adhesive composite and release liner from the bottom packaging sheet; securing a portion of the medical adhesive composite to the skin of a patient, wherein securing comprises contacting the skin of the patient with at least some of the portion of the pressure sensitive adhesive layer not in contact with the release liner; and securing the medical adhesive composite to the skin of the patient, wherein securing comprises simultaneously separating the release liner from the medical adhesive composite and increasing contact between the skin and pressure sensitive adhesive layer formerly in contact with the release liner.

### BRIEF DESCRIPTION OF DRAWINGS

The invention may be more completely understood in consideration of the following detailed description in connection with the following figures:

FIGS. 1a and 1b are schematic cross-sectional views of exemplary packaged medical adhesive composites.

FIGS. 2a and 2b show perspective views of an exemplary medical adhesive composite being separated from packaging.

FIG. 2c shows a plan view of the exemplary medical adhesive composite shown in FIGS. 2a and 2b.

FIGS. 3a-3c show delivery of an exemplary medical adhesive composite with only a single hand.

### DETAILED DESCRIPTION

Various constructions of medical adhesive composites are known, and many of these constructions are aimed at delivering a composite having an especially thin supple backing to the skin of a patient, without wrinkling of the backing, or the backing sticking to itself. Deformation of a thin backing can be particularly problematic if one does not want to touch the sterile underside of the composite before it is applied. Many medical adhesive composites have traditional two-tab constructions which make handling difficult after the tabs are removed. Carrier frames and carrier backings have been used to facilitate delivery of medical adhesive composites, however, they can delaminate, tear or lift the composite from the skin upon removal from the composite backing. Further, they do not allow a user to overlap the composite upon itself when applied, for example, to a finger.

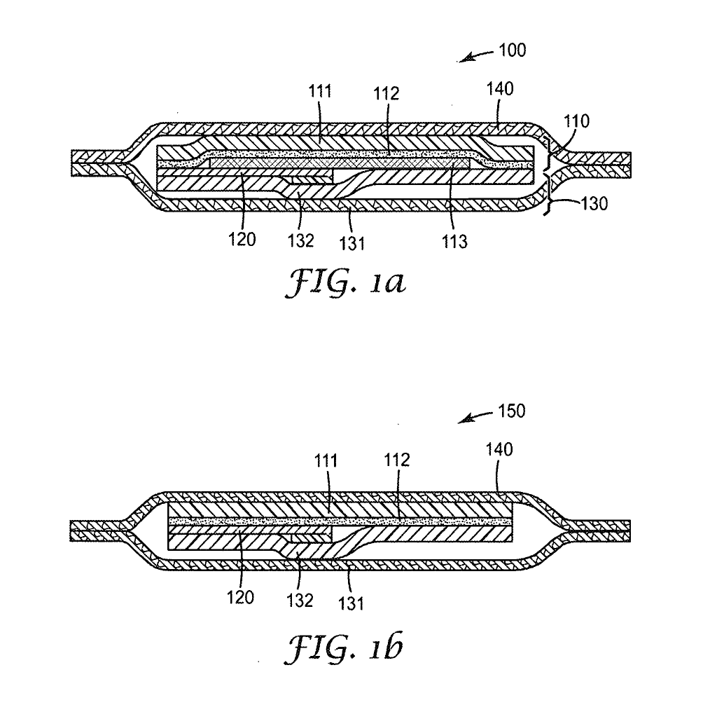

FIG. 1a is a schematic cross-sectional view of an exemplary packaged medical adhesive composite. Packaged composite 100 comprises medical adhesive composite 110 comprising backing 111, pressure sensitive adhesive layer 112 disposed on the backing, and absorbent pad 113 adhered to the backing by the pressure sensitive adhesive layer. Packaged medical adhesive composite 100 further comprises release liner 120 disposed on a portion of pressure sensitive adhesive layer 112 opposite the backing, such that a portion of the pressure sensitive adhesive layer is not adjacent the release liner. Exemplary packaged medical adhesive composite 100 further comprises packaging comprising bottom packaging sheet 130 and top packaging sheet 140. Bottom packaging sheet 130 comprises bottom substrate 131 and release layer 132 disposed on the bottom substrate, and the release layer is in contact with release liner 120 and pressure sensitive adhesive layer 112. Top packaging sheet 140 and bottom packaging sheet 130 are sealed together about the periphery of medical adhesive composite 110 and release liner 120.

FIG. 1b is a schematic cross-sectional view of an exemplary packaged medical adhesive composite. Packaged composite 150 comprises packaged medical adhesive composite 100 except that absorbent pad 113 is not present.

The backing may comprise any type of film, paper or fabric used in making medical adhesive composites and is selected depending on desired properties of the composite. Typically, the medical adhesive composite is applied such that the absorbent pad is over a wound and the pressure sensitive adhesive layer adheres the composite to the skin of a patient. Preferably, the backing is conformable, i.e., thin, flexible and supple, such that it conforms to the contours of a patient's skin. The backing desirably has moisture vapor permeability (MVP) of at least about 300 g/m²/24 hrs/37° C./100-10% relative humidity as measured according to the inverted cup method described in U.S. Pat. No. 4,595,001 (Potter et al.), which is greater than that of human skin.

Representative backings include nonwoven fibrous webs, woven fibrous webs, knits, foams and films such as polymeric films. The backing may comprise a polymeric film that is transparent and/or translucent. The backing is conformable to anatomical surfaces and joints even when the surface or joint is moved. When a joint is flexed and returned to its unflexed position, the backing should stretch to accommodation the flexion of the joint, but at the same time is resilient enough to continue to conform to the joint when the joint is returned to its unflexed condition. Exemplary polymeric films that may be used as the backing include elastomeric films made from polyurethanes, polyesters, polyether block amides, or combinations thereof. Additional polymeric films that may be used are described in U.S. Pat. No. 5,088,483 (Heinecke) and U.S. Pat. No. 5,160,315 (Heinecke et al.).

The pressure sensitive adhesive layer may comprise any type of pressure sensitive adhesive layer used in making medical adhesive composites and is selected depending on desired properties of the composite. The pressure sensitive adhesive layer should be able to adhere the composite to the skin of a patient, over anatomical surfaces and joints even when flexed as described above, yet the layer should permit removability of the composite without harming the skin and/or wound. The bond strength between the pressure sensitive adhesive layer and the backing should be great enough such that the two do not separate under normal use, even when the composite is being removed from a patient. The pressure sensitive adhesive layer may be selected such its MVP is at least that of the backing as described above. Preferably, the pressure sensitive adhesive layer is safe to use on human and/or animal skin and may be specifically a hypoallergenic layer.

The pressure sensitive adhesive layer may comprise a (meth)acrylate copolymer, e.g., an iso-octylacrylate/acrylamide copolymer or an iso-octylacrylate/ethylene oxide acrylate/acrylic acid copolymer. Additional pressure sensitive adhesive layers that may be used are described in U.S. Pat. No. 3,389,827 (Abere et al.), U.S. Pat. No. 4,112,213 (Waldman), U.S. Pat. No. 4,310,509 (Berglund et al.), U.S. Pat. No. 4,323,557 (Rosso et al.), U.S. Pat. No. 4,737,410 (Kantner) and U.S. Re. 24,906 (Ulrich). Addenda such as medicaments and antimicrobial agents may be incorporated in the pressure sensitive adhesive layer as described in Berglund et al. and Rosso et al. The pressure sensitive adhesive layer may be patterned as described in U.S. Pat. No. 4,595,001 (Potter et al.).

The pressure sensitive adhesive layer is disposed on the backing, and the layer may or may not completely cover the backing, depending on the design of the medical adhesive composite. Preferably, the pressure sensitive adhesive layer completely covers the backing such that the surface of the backing to which the pressure sensitive adhesive is applied is not exposed.

The absorbent pad is adhered to the backing on the same side as the pressure sensitive adhesive layer. As shown in FIG. 1a, the backing may be adhered to the backing by the pressure sensitive adhesive layer, e.g., the absorbent pad may be applied to the pressure sensitive adhesive layer opposite the backing. Any other suitable material may be used to adhere the absorbent pad to the backing. It is desirable that the absorbent pad be adhered to the backing such that the pad does not separate from the backing during use or removal of the medical adhesive composite from a patient.

The absorbent pad is suitable for use over a wound and may comprise woven or nonwoven cotton, rayon, nonwovens, hydrocolloids, foams or combinations thereof The absorbent pad may comprise antimicrobial agents, drugs for transdermal drug delivery, chemical indicators to monitor substances or combinations thereof.

A optional polymeric layer may be disposed on the backing opposite the pressure sensitive adhesive layer. This optional layer may be used to modify the top surface of the backing, for example, the layer may be used to provide or protect an image or graphic on or part of the backing. An exemplary optional polymeric layer comprises polyvinyl N-octadecyl carbamate and a blend of silicone resins as described in U.S. Pat. No. 5,738,642 (Heinecke et al.).

A release liner is disposed on a portion of the pressure sensitive adhesive layer opposite the backing, i.e., the release liner is disposed only on a portion of the pressure sensitive adhesive layer such that a portion of the pressure sensitive adhesive layer is not adjacent or in contact with the release liner. The portion of the pressure sensitive adhesive layer that is not adjacent or in contact with the release liner may begin at an opposing end of the pressure sensitive adhesive layer and extend towards the release liner as shown in FIGS. 1, 2 and 3a-3c. When the medical adhesive composite with release liner are removed from the bottom packaging sheet, enough of the pressure sensitive adhesive layer is exposed so that a user can adhere the medical adhesive composite to the skin of a patient, and the medical adhesive composite stays in place upon removal of the release liner as described below. The amount of exposed pressure sensitive adhesive layer depends on a variety of factors such as the width of the layer, the bond strength of the pressure sensitive adhesive layer to the skin, the size and shape of the release liner to be removed, etc. For example, the pressure sensitive adhesive layer not in contact with the release liner may comprise from about 5 to about 80% of the area of the pressure sensitive adhesive layer, from about 5 to about 70% of the pressure sensitive adhesive layer, from about 10 to about 70% of the area of the pressure sensitive adhesive layer, or from about 25 to about 60% of the area of the pressure sensitive adhesive layer.

The release liner may partially or completely cover the absorbent pad. In some cases, the release liner does not cover the absorbent pad.

Generally, the release liner may have any shape and/or size that is useful for the delivery of the medical adhesive composite. The release liner may be partially commensurate in shape and size with the backing, it may be substantially the same shape as the backing but extend slightly beyond the periphery of the backing, or it may be substantially the same shape as the backing and extend close to the periphery of the backing but not beyond it.

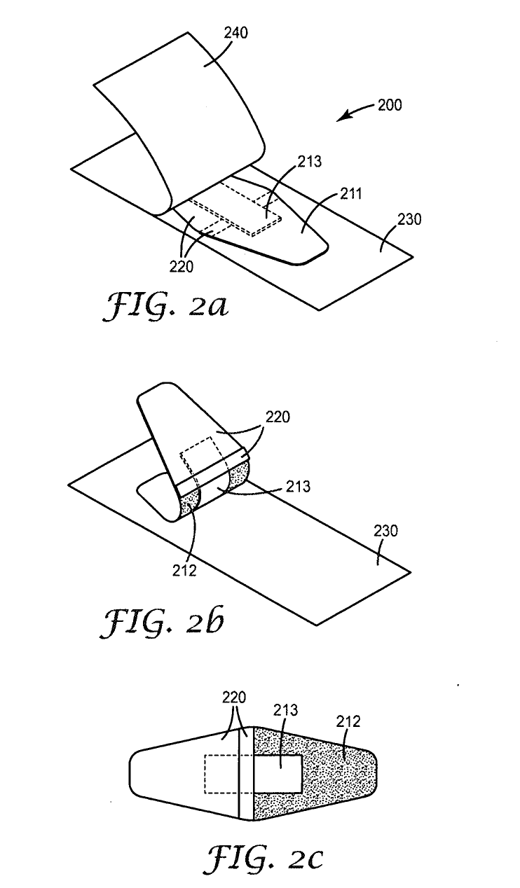

The portion of the release liner may be folded back upon itself as shown in FIGS. 1a and 1b for release liner 120. FIGS. 2a and 2b show perspective views of an exemplary medical adhesive composite being separated from packaging. In FIG. 2a, top packaging sheet 240 has been separated from bottom packaging sheet 230 and is shown being pulled away from the composite and bottom packaging sheet 230. The medical adhesive composite comprises backing 211 with PSA layer 212 disposed on the backing and absorbent pad 213 disposed on the PSA layer. Release liner 220 is disposed on the PSA layer opposite the backing. Once bottom packaging sheet 230 is removed, the medical adhesive composite is pulled away from bottom packaging sheet 230 as shown in FIG. 2b. FIG. 2c shows a plan view of the medical adhesive composite shown in FIGS. 2a and 2b, with release liner 220 and PSA layer 212 facing up.

The release liner is releasably adhered to the pressure sensitive adhesive layer such that it can be released or removed from the pressure sensitive adhesive layer without undesirably affecting integrity of the pressure sensitive adhesive layer. Typically, this means that the bond strength between the pressure sensitive adhesive layer and the release liner is less than that between the pressure sensitive adhesive layer and the backing. The release liner is more rigid than the combination of the backing and the pressure sensitive adhesive layer.

In some cases, it is desirable that the bond strength between the release liner and the pressure sensitive adhesive layer be great enough such that the liner can facilitate application of the composite, for example, by providing sufficient support for the backing while the liner is being pulled away from the pressure sensitive adhesive layer during application of the composite.

Release liners which may be used are well known and typically comprise paper or polymer film coated with release agents such as silicone materials or fluorochemicals. The particular release liner used depends on the properties of the pressure sensitive adhesive layer as well as desired release characteristics between the release liner and the pressure sensitive adhesive layer. Useful release liners and test methods that aid selection of release liners are described in U.S. Pat. No. 6,264,976 B1 (Heinecke et al.), U.S. Pat. No. 5,738,642 (Heinecke et al.) and WO 98/00080 (Dunshee et al.).

The medical adhesive composite with release liner is packaged with packaging comprising top and bottom packaging sheets that are sealed together about the periphery of the composite and the liner. Generally, the bottom packaging sheet has a larger area than the medical adhesive composite and release liner, such that the bottom packaging sheet, with medical adhesive composite and release liner disposed on it, is observable around all sides of the medical adhesive composite and release liner combination when viewed from above the medical adhesive composite.

The bottom packaging sheet comprises a bottom substrate and a release layer disposed on the bottom substrate. The release layer is in contact with the pressure sensitive adhesive layer opposite the backing, i.e., the release layer is in contact with the portion of the pressure sensitive adhesive layer that is not in contact with the release liner. The release layer is in contact with enough of the exposed pressure sensitive adhesive layer not covered by the release liner, such that the medical adhesive composite can be release from the bottom packaging sheet without damaging the composite. The release layer may or may not completely cover the bottom substrate as long as the layer does not interfere with sealing about the periphery of the top and bottom packaging sheets. Preferably, the periphery of the bottom packaging sheet to be sealed with the top packaging sheet does not have release layer disposed on it. The release layer may have an area commensurate in shape and size of the backing and release liner. The release layer may also have an area commensurate with the shape and size of the pressure sensitive adhesive layer not in contact with the release liner.

The release layer may comprise a release agent coated on the bottom substrate, with or without any intermediate layers. The release layer may also comprise a bottom release liner that is attached to the bottom substrate using a material such as an adhesive or the sealable material used to seal the top and bottom packaging sheets as described below. The bottom release liner may also be attached by some mechanical means to the bottom packaging sheet.

Release agents used for the release layer are well known and include silicone materials and fluorochemicals. Release liners used as the bottom release liner are well known and typically comprise paper or polymer film coated with release agents such as silicone materials or fluorochemicals. The particular release liner used depends on the properties of the pressure sensitive adhesive layer as well as desired release characteristics between the release liner and the pressure sensitive adhesive layer. Useful release liners and test methods that aid selection of release liners are described in U.S. Pat. No. 6,264,976 B1 (Heinecke et al.), U.S. Pat. No. 5,738,642 (Heinecke et al.) and WO 98/00080 (Dunshee et al.).

Exemplary bottom substrates include paper, wovens, nonwovens and polymeric films such as polyethylene, polypropylene, polyester, etc., or any other substrate that can be used to make a suitable package. Additional bottom substrates are described in U.S. Pat. No. 6,099,682 (Krampe et al.)

The top packaging sheet is disposed on the backing opposite the pressure sensitive adhesive layer. Generally, the top packaging sheet has a larger area than the medical adhesive composite and release liner, such that the top packaging sheet, with medical adhesive composite and release liner disposed on it, is observable around all sides of the medical adhesive composite and release liner combination when viewed from below. Exemplary top packaging sheets comprise paper, wovens, nonwovens and polymeric films such as polyethylene, polypropylene, polyester, etc. or any other sheet that can be used to make a suitable package. The top packaging sheet may comprise one or more layers such as coated papers and coated polymeric films. Additional bottom substrates are described in Krampe et al.

The top and bottom packaging sheets comprise inner faces that face each other when the package is assembled. One or both of the inner faces may have a sealable material disposed on it; the sealable material can facilitate sealing about the periphery of the substrates. The sealable material may be present as a layer disposed on the entire surface of a top or bottom packaging sheet, or it may be present around the periphery only of the top and/or bottom packaging sheets. Cold seal packaging as described in Krampe et al. may be employed and utilizes contact adhesives such as latex rubber and contact adhesives substantially free of latex rubber including polychloroprenes, polyurethanes, styrene-isoprene copolymers, styrene-butadiene copolymers, polyimides, polyvinylchlorides and poly(meth)acrylates. Pressure sensitive adhesives may be used.

One or both of the top and bottom packaging sheets may be unattached along one edge to form tabs that facilitate separation of the top and bottom packaging sheets.

The medical adhesive composite may be made by any number of manufacturing methods disclosed in the references cited herein.

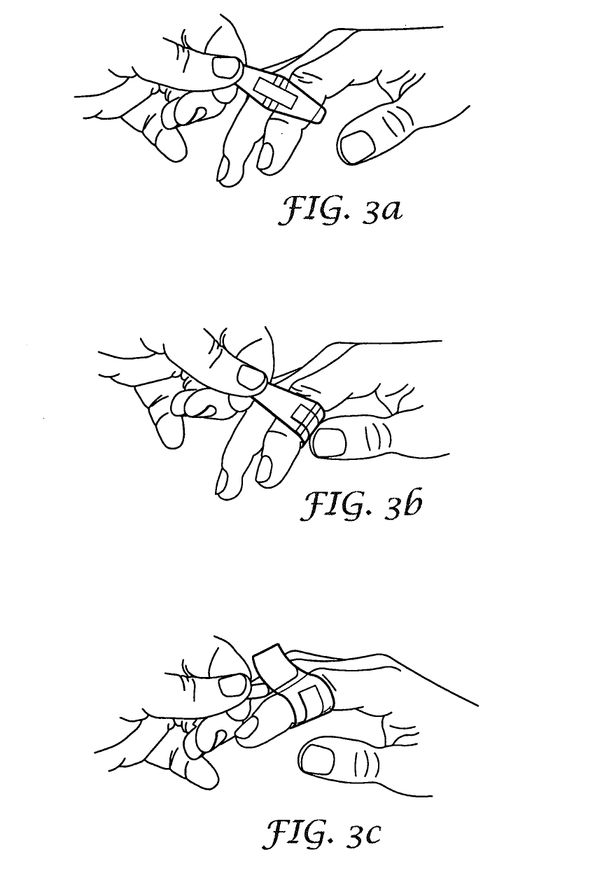

FIGS. 3a-3c show an exemplary method for delivering or applying an exemplary medical adhesive composite over a wound on a patient's finger. The method comprises providing the packaged medical adhesive composite and at least partially separating the top and bottom packaging sheets so that the medical adhesive composite with attached release liner can be separated from the bottom packaging sheet. Upon separating, the portion of the pressure sensitive adhesive layer not in contact with the release liner is exposed. A next step is shown in FIG. 3a wherein a portion of the exposed pressure sensitive adhesive layer is contacted with the skin, followed by the step shown in FIG. 3b wherein the exposed pressure sensitive adhesive layer is more fully contacted with the skin such that the medical adhesive composite is secured to the skin. The remainder of the medical adhesive composite is secured to the skin of the patient as shown in FIG. 3c wherein the end of the release liner that is close to the center of the medical adhesive composite is pulled away from the PSA layer while simultaneously using the release liner to “wipe” down the composite on the finger. The steps shown in FIGS. 3a-3c can be carried out with a single hand.

What is claimed is:

1. A packaged medical adhesive composite comprising:
(a) a medical adhesive composite comprising:
a backing, and
a pressure sensitive adhesive layer disposed on the backing;
(b) a release liner disposed on a portion of the pressure sensitive adhesive layer opposite the backing, such that a portion of the pressure sensitive adhesive layer is not in contact with the release liner; and
(c) packaging comprising:
a bottom packaging sheet comprising a bottom substrate and a release layer disposed on the bottom substrate, wherein the portion of the pressure sensitive adhesive layer not in contact with the release liner is in contact with the release layer; and
a top packaging sheet disposed on the backing opposite the pressure sensitive adhesive layer, wherein the top and bottom packaging sheets are sealed together about the periphery of the medical adhesive composite and the release liner.

2. The packaged medical adhesive composite of claim 1, wherein the portion of the pressure sensitive adhesive layer not in contact with the release liner comprises from about 5 to about 70% of the area of the pressure sensitive adhesive layer.

3. The packaged medical adhesive composite of claim 1, wherein the portion of the pressure sensitive adhesive layer not in contact with the release liner comprises from about 25 to about 60% of the area of the pressure sensitive adhesive layer.

4. The packaged medical adhesive composite of claim 1, the medical adhesive composite further comprising an absorbent pad disposed on the pressure sensitive adhesive layer opposite the backing.

5. The packaged medical adhesive composite of claim 4, wherein the release liner is disposed on a portion of the absorbent pad.

6. The packaged medical adhesive composite of claim 1, wherein the top and bottom packaging sheets are sealed together with a contact adhesive comprising latex rubber.

7. The packaged medical adhesive composite of claim 1, wherein the top and bottom packaging sheets are sealed together with a contact adhesive that is substantially free of latex rubber.

8. A method of delivering a medical adhesive composite over a wound, comprising:
providing a packaged medical adhesive composite comprising:
(a) a medical adhesive composite comprising:
a backing, and
a pressure sensitive adhesive layer disposed on the backing;
(b) a release liner disposed on a portion of the pressure sensitive adhesive layer opposite the backing, such that a portion of the pressure sensitive adhesive layer is not in contact with the release liner; and
(c) packaging comprising:
a bottom packaging sheet comprising a bottom substrate and a release layer disposed on the bottom substrate, wherein at least some of the portion of the pressure sensitive adhesive layer not in contact with the release liner is in contact with the release layer; and
a top packaging sheet disposed on the backing opposite the pressure sensitive adhesive layer, wherein the top and bottom packaging sheets are sealed together about the periphery of the medical adhesive composite and the release liner;
(ii) at least partially separating the top and bottom packaging sheets;
(iii) separating the medical adhesive composite and release liner from the bottom packaging sheet;
(iv) securing a portion of the medical adhesive composite to the skin of a patient, wherein securing comprises contacting the skin of the patient with at least some of the portion of the pressure sensitive adhesive layer not in contact with the release liner; and
(v) securing the medical adhesive composite to the skin of the patient, wherein securing comprises simultaneously separating the release liner from the medical adhesive composite and increasing contact between the skin and pressure sensitive adhesive layer formerly in contact with the release liner.

9. The method of claim 8, wherein the medical adhesive composite is applied with only a single hand.
